(19) United States
(12) Patent Application Publication
(10) Pub. No.: US 2004/0019287 A1
(43) Pub. Date: Jan. 29, 2004

(54) Similarity recovery post shock

(76) Inventor: Harley White, Carnation, WA (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/206,798

(22) Filed: Jul. 26, 2002

(51) Int. Cl. A61N 1/362

(52) U.S. Cl. 600/509; 607/9

(57) ABSTRACT

A cardiac rhythm management system adapted to administer therapy based on identification and analysis of a trend in a series of similarity metrics based on a comparison of a cardiac complex and a template at a time following an electrical stimulation. Where the trend indicates a monotonically increasing correlation, therapy is inhibited and otherwise, therapy is delivered. In one embodiment, analysis of the trend includes discarding spurious data, executing a smoothing function, and calculating a moving average.

### TECHNICAL FIELD

This invention relates generally to cardiac rhythm management systems and particularly, but not by way of limitation, to a method of administering therapy.

### BACKGROUND

Effective, efficient ventricular pumping action depends on proper cardiac function. Proper cardiac function, in turn, relies on the synchronized contractions of the heart at regular intervals. When normal cardiac rhythm is initiated at the sinoatrial node, the heart is said to be in normal sinus rhythm (NSR) or sinus rhythm. However, when the heart experiences irregularities in its coordinated contraction, due to electrophysiological disturbances caused by a disease process or from an electrical disturbance, the heart is said to be arrhythmic. The resulting cardiac arrhythmia impairs cardiac efficiency and can be potentially life threatening.

A cardiac arrhythmia occurring in the atrial of the heart is called a supraventricular tachyarrhythmia (SVT) and a cardiac arrhythmia occurring in the ventricular region of the heart is called a ventricular tachyarrhythmia (VT). SVTs and VTs are electrically (morphologically and physiologically) distinct events. VTs take many forms, including ventricular fibrillation (VF) and ventricular tachycardia. Ventricular fibrillation is a condition denoted by extremely rapid, nonsynchronous contractions of the ventricles. This condition is fatal unless the heart is returned to sinus rhythm within a few minutes. Ventricular tachycardia are conditions denoted by a rapid heart beat, 150 to 250 beats per minute, that has its origin in some abnormal location within the ventricular myocardium. The abnormal location may result from damage to the ventricular myocardium caused by a myocardial infarction. Ventricular tachycardia can quickly lead to ventricular fibrillation.

SVTs also take many forms, including atrial fibrillation and atrial flutter. Both conditions are characterized by rapid uncoordinated contractions of the atria. Besides being hemodynamically inefficient, the rapid contractions of the atria can also adversely effect the ventricular rate. This occurs when the aberrant contractile impulses in the atria are transmitted to the ventricles.

Implantable cardioverter/defibrillators (ICDs) have been used to treat patients with serious ventricular tachyarrhythmias. Some ICDs are able to recognize and treat tachyarrhythmias with a variety of tiered therapies. These tiered therapies range from providing antitachycardia pacing or cardioversion energy for treating tachycardia to defibrillation energy for treating ventricular fibrillation.

To effectively deliver treatment, an ICD must first identify the type of tachyarrhythmia occurring in the heart. Attempts at identifying tachyarrhythmias have included comparing the morphologies of individual cardiac complexes to model, or template, cardiac complexes. Template cardiac complex morphologies are created from cardiac complexes sensed from a single channel or multiple channel electrogram. Once created, the template cardiac complex morphologies are integrated into morphology algorithms programmed into the ICD. As the ICD encounters a tachycardia episode, cardiac complexes sensed on the single channel electrogram are compared to the template cardiac complex morphologies in the morphology algorithms. The morphology exhibited by an electrogram is a function of the originating location of the tachycardia.

Exemplary systems and methods for classifying a cardiac complex are presented in commonly assigned U.S. patent application Ser. No. 09/848,605, filed May 3, 2001, entitled SYSTEM AND METHOD FOR CLASSIFYING CARDIAC COMPLEXES, inventors William Hsu et al., and which is a continuation of U.S. Pat. No. 6,266,554, issued Jul. 24, 2001, filed Feb. 12, 1999, and also entitled SYSTEM AND METHOD FOR CLASSIFYING CARDIAC COMPLEXES, inventors William Hsu et al. The specifications of U.S. patent application Ser. No. 09/848,605 and U.S. Pat. No. 6,266,554, are herein incorporated by reference.

Transient, post-shock changes in electrogram morphology may complicate delivery of suitable cardiac therapy. For example, for a period of time soon after delivery of a shock, the cardiac morphology may exhibit low correlation with a template cardiac complex. Additional shocks delivered during the transient period may further aggravate or deteriorate the patient's condition.

ICDs, also referred to as cardiac rhythm management systems, include cardioverters or defibrillators that are capable of delivering higher energy electrical stimuli to the heart. Defibrillators are often used to treat patients with tachyarrhythmias, that is, hearts that beat too quickly. Such too-fast heart rhythms also cause diminished blood circulation because the heart is not allowed sufficient time to fill with blood before contracting to expel the blood. Such pumping by the heart is inefficient. A defibrillator is capable of delivering a high energy electrical stimulus that is sometimes referred to as a defibrillation countershock. The countershock interrupts the tachyarrhythmia, allowing the heart to reestablish a normal rhythm for the efficient pumping of blood. In addition to pacers, cardiac rhythm management systems also include, among other things, pacer/defibrillators that combine the functions of pacers and defibrillators, drug delivery devices, and any other systems or devices for diagnosing or treating cardiac arrhythmias.

For the reasons stated above, and for other reasons which will become apparent to those skilled in the art upon reading and understanding the present specification, there is a need in the art for providing a reliable system and method for analyzing cardiac complexes and delivering therapy.

### SUMMARY

This document discloses, among other things, a cardiac rhythm management system which assists in the administration of electrical therapy for the treatment of a heart condition. In one embodiment, a template corresponding to NSR is compared with a cardiac complex at a time after a shock has been delivered to the heart. The shock disturbs the morphology of the cardiac complex. Analysis of the cardiac complex relative to the template may reveal that the heart is responding favorably or unfavorably to the delivered shock. In the event that the heart is responding favorably, then a subsequent shock may frustrate the recovery and yet further aggravate the condition of the heart. If, on the other hand, the heart is not responding favorably to the shock, then the present subject may be programmed to administer additional therapy which may include delivery of a subsequent shock.

Various metrics may be applied to classify the heart's response to the shock as favorable or unfavorable. In one embodiment, the classification is based on a moving average correlation coefficient. If the correlation coefficient is trending towards higher correlation, then the heart is considered to be responding favorably and shock therapy is inhibited. In one embodiment, a monotonically increasing correlation coefficient denotes a favorable response by the heart. Conversely, a correlation coefficient not increasing monotonically does not inhibit therapy. In one embodiment, a minimum rate of change, or derivative, of the correlation coefficient is sufficient to inhibit therapy. Other metrics may also be applied in administering therapy. As used herein, administering therapy includes both delivering therapy and inhibiting therapy.

For example, in one embodiment, if three of the ten most recent heart beats in the cardiac complex exhibit correlation with the template in excess of 94% correlation, then therapy is inhibited. Values other than three out of ten and 94% may also be used.

In one embodiment, the present subject matter includes an analysis of a trend over a series of cardiac complexes. Therapy is delivered or inhibited based on analysis of any trend. For example, in one embodiment, if three of ten cardiac complexes are correlated to within 96%, then the present subject matter inhibits therapy. If eight of the last ten cardiac complexes are not correlated to within 94%, then evaluate any trend of the series of correlations.

In one embodiment, a correlation coefficient relates cardiac complexes, or heart beats, to a template. Correlation, in one embodiment, is expressed in percentages and provides a measure of the similarity between the cardiac complex and the template.

In one embodiment, a correlation measure is calculated which represents an average of the ten most recent cardiac complexes. The correlation measure typically falls between zero and 100%. In one embodiment, the previous ten cardiac complexes are evaluated to determine if any eight are correlated to a level greater than, or less than, 96 percent.

In one embodiment, a moving average is calculated for each beat based on the last cardiac complex and the nine prior cardiac complexes. The moving average provides a measure of average similarity. In one embodiment, if the new average similarity measure exceeds the prior average similarity measure for eight out of ten measures, then inhibit therapy.

Other aspects of the invention will be apparent on reading the following detailed description of the invention and viewing the drawings that form a part thereof.

### BRIEF DESCRIPTION OF THE DRAWINGS

In the drawings, like numerals describe substantially similar components throughout the several views. Like numerals having different letter suffixes represent different instances of substantially similar components.

FIG. 1A includes a portion of a flow chart according to one embodiment of the present subject matter.

FIG. 1B includes a graphical depiction of sample data and calculations according to one embodiment of the present subject matter.

FIG. 2 includes a block diagram illustrating generally one embodiment of portions of a cardiac rhythm management system.

FIG. 3 illustrates a block diagram of a portion of a cardiac rhythm management system.

FIG. 4A illustrates a portion of a flow chart according to one embodiment of the present subject matter.

FIG. 4B illustrates a portion of a flow chart according to one embodiment of the present subject matter.

FIG. 4C illustrates a portion of a flow chart according to one embodiment of the present subject matter.

FIG. 5 illustrates a cardiac complex and a template.

FIG. 6 illustrates a schematic diagram of an implantable cardiac device electrically coupled to a heart.

FIG. 7 graphically illustrates a model of correlation as a function of time for a first selected series of cardiac complexes.

FIG. 8 graphically illustrates a model of correlation as a function of time for a second selected series of cardiac complexes.

FIG. 9 graphically illustrates a model of correlation as a function of time for a third selected series of cardiac complexes.

FIG. 10 illustrates a portion of a flow chart executed by one embodiment of the present subject matter.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, logical and electrical changes may be made without departing from the spirit and scope of the present invention. The following detailed description is, therefore, not to be taken in a limiting sense, and the scope of the present invention is defined by the appended claims and their equivalents. In the drawings, like numerals describe substantially similar components throughout the several views. Like numerals having different letter suffixes represent different instances of substantially similar components.

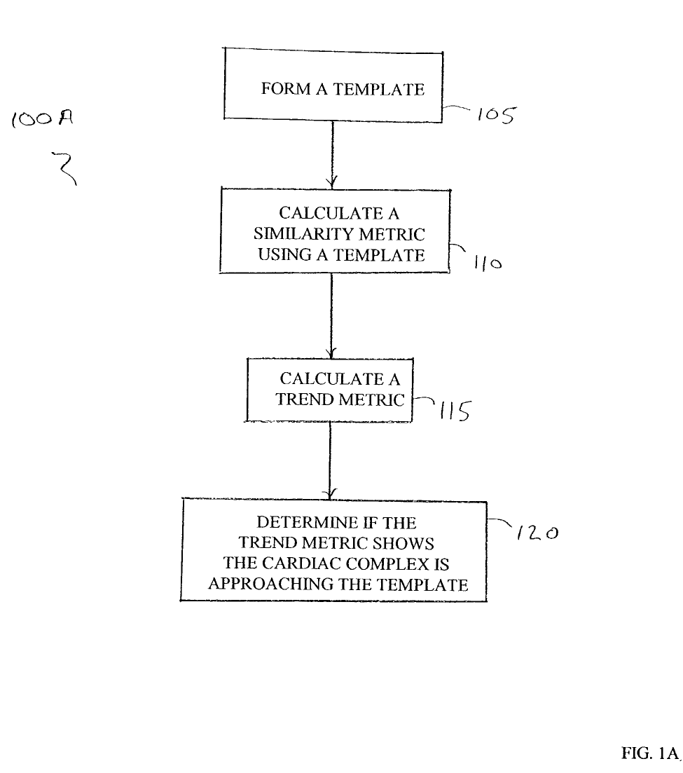

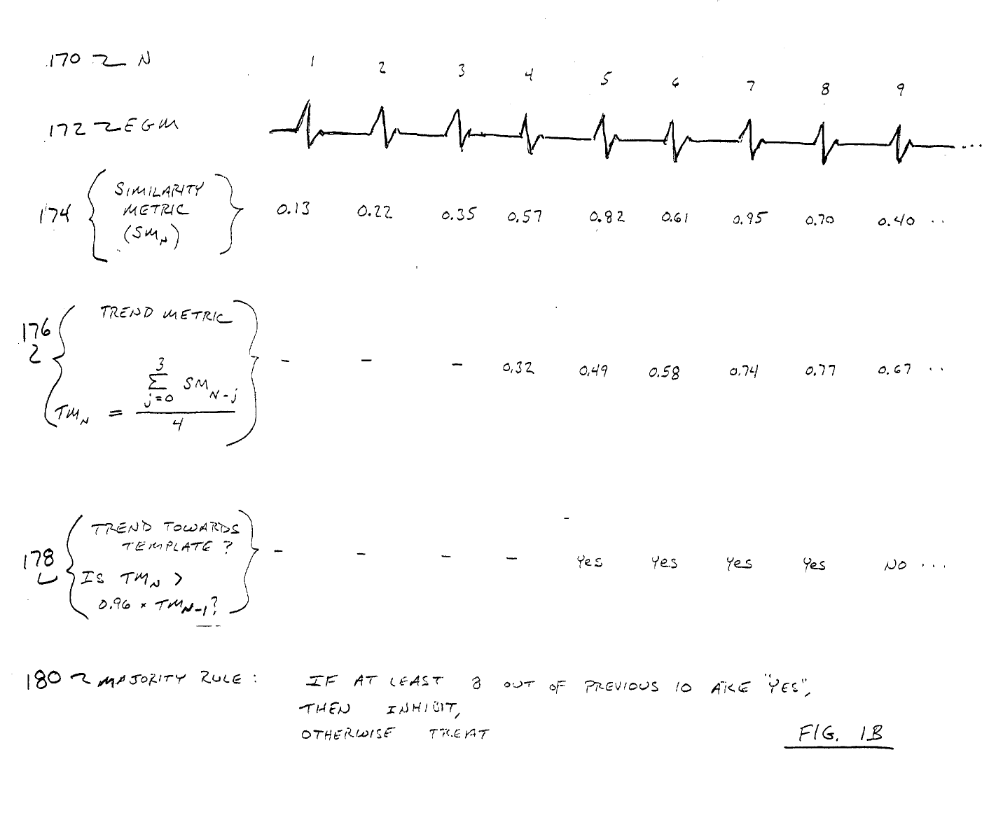

The flow chart in FIG. 1A illustrates method 100A according to one embodiment of the present subject matter. Method 100A is a portion of a method to be executed by an implantable device or an external device, and in one embodiment, the method is implemented by a processor executing a set of instructions. In the method, a template is formed, or received, at 105. The template may be derived from historical data for a particular user, or patient, or the template may be derived from model data. The template may be received from a source external to the implanted device. In one embodiment, the template is stored in a memory accessible to a processor adapted for executing method 100A.

The template corresponds to a normal rhythm. The template may include data for a super ventricular rhythm or a sinus tachycardia rhythm.

At 110, an unknown cardiac complex is compared with the template to calculate a similarity metric. The cardiac complex typically includes one or more heart beats. In this context, the unknown cardiac complex follows delivery of an electrical stimulation and may be associated with an atrial tachycardia or ventricular tachycardia episode. The electrical stimulation may have been delivered by an ICD, pulse generator or other device and may be administered to treat a bradycardia, fibrillation, to achieve synchronization of the heart chambers or to treat other electrophysiological conditions of the heart. The electrical stimulation, in one embodiment, includes delivery of a therapeutic shock. In one embodiment, at 110, a percentage match or correlation is calculated. This reflects a degree to which the cardiac complex is similar to the template.

In one embodiment, the comparison of the unknown cardiac complex with the template results in the calculation of a correlation coefficient or other similarity metric.

At 115, method 100A proceeds by calculating a trend metric based on a portion, or subset, of the series of similarity metrics. In one embodiment, calculation of a trend metric includes calculating an average correlation. In one embodiment, each successive heart beat results in a correlation coefficient. In one embodiment, rather than calculate a mathematical average correlation coefficient, a metric based on the correlation coefficient is developed, which aids in the analysis of the trend exhibited by the cardiac complex.

A variety of mathematical techniques may be applied to determine whether the series of cardiac complexes is trending towards the template, and therefore, responding favorably to the shock. For example, in one embodiment, a nonparametric function estimator, or smoothing function is used to approximate the data. In one embodiment, a maximum high correlation coefficient and a minimum low correlation coefficient are discarded before calculating an average. Based on considerations of estimated efficiency and computational demands, a smoothing function or other trend analysis routine may be utilized.

Referring again to 115 of FIG. 1A, one embodiment includes calculation of a moving average correlation coefficient.

At 120, the method includes application of a rule to determine if the trend metric shows the cardiac complex is approaching the template. In one embodiment, this analysis calls for discerning a monotonically increasing trend. In one embodiment, if the metric is decreasing (generally or monotonically), indeterminate (or relatively unchanged) or increasing generally (but not monotonically), then therapy is delivered and if the metric is increasing monotonically, then therapy is inhibited.

The method performed by the present subject matter may be expressed in different terms. For example, rather than inhibiting therapy for a monotonically increasing series of correlation coefficients, in one embodiment, therapy is delivered if the series of correlation coefficients does not monotonically increase.

In one embodiment, the presence or absence of a monotonic increase is used to determine administration of electrical therapy. Other markers can also be monitored. For example, in one embodiment, an algorithm is adapted to determine if the series of correlation coefficients indicates that the cardiac complex is approaching, or asymptotically approaching, the template morphology.

FIG. 1B graphically illustrates a series of nine sample heart beats, or cardiac complexes, and related calculations corresponding to determining if therapy should be inhibited or delivered. In the figure, row 172 illustrates a sample electrocardiogram (EGM, ECG or EKG) series of consecutive beats for a particular heart and row 170 identifies the heart beat number where each beat is labeled variable N.

In row 174, similarity metric SM_{N} is determined by comparing heart beat N with a template. For example, for N=1, SM_{N} is 0.13 and for N=5, SM_{N}=0.82. The value of each similarity metric ranges between zero and one and in some contexts, is referred to as feature correlation coefficient, or FCC. In row 176, a trend metric TM_{N} is calculated by determining the average of the previous four similarity metrics. At N=4, TM_{N} is calculated by averaging the similarity metrics for N=1, 2, 3 and 4 and in this case, that calculation reduces to 1/4(0.13+0.22+0.35+0.57), thus TM_{N}=0.32. For N=1, 2 and 3, insufficient data precludes calculation of a trend metric. In one embodiment, the trend metric is a moving average.

In row 178, a calculation is performed to identify a trend towards the template. In one embodiment, the calculation includes determining if the previous TM_{N} is greater than 96% of the previous TM_{N}. Expressed mathematically, the calculation reduces to determining if TM_{N}>(0.96)(TM_{N-1})? Thus, for example, at N=5, 0.49 is greater than 96% of 0.32, and thus, the trend is affirmatively towards the template. At N=9, on the other hand, the trend is not towards the template since 0.67 is not greater than 96% of 0.77. For N=1, 2, 3 and 4, insufficient data precludes determining if heartbeats in the series are trending towards the template. Values other than a multiplier of 96% can also be used with lower multiplier values softening the trend required before inhibiting therapy and higher multiplier values sharpening the trend requirement.

To determine if therapy should be inhibited or delivered, row 180 presents a rule to decide if at least eight values of N out of the previous ten values of N are marked with a yes answer to the inquiry of row 178. If application of the rule in row 180 indicates that a “yes” appears with eight of ten prior heart beats, then therapy is inhibited. If not, then therapy is delivered.

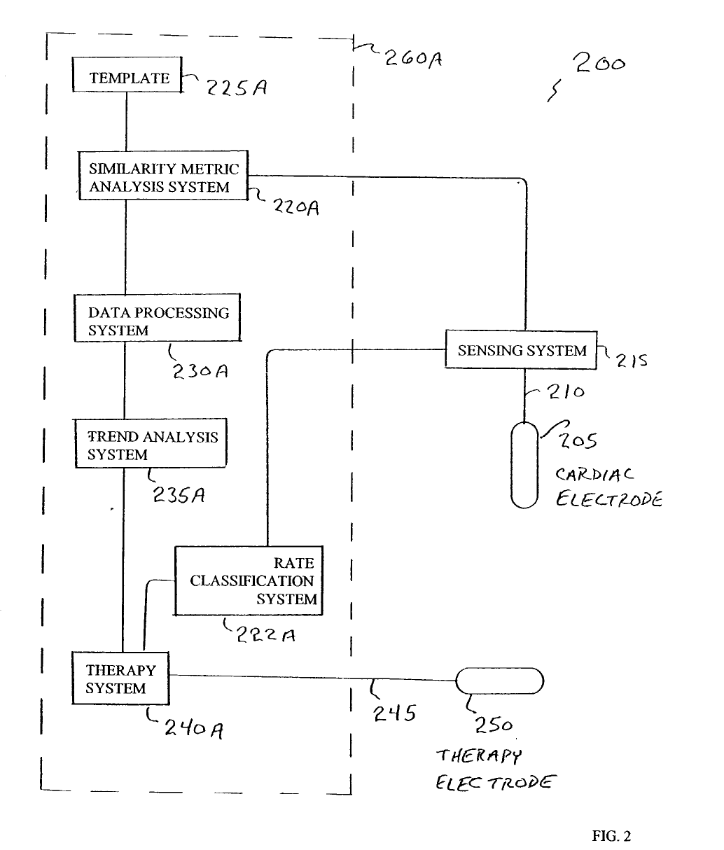

In FIG. 2, implanted device 200 is illustrated. In the figure, cardiac electrode 205 is coupled by lead 210 to sensing system 215. Sensing system 215 is further coupled to similarity metric analysis system 220A and rate classification system 222A. Similarity metric analysis system 220A is communicatively coupled to template 225A and data processing system 230A. Data processing system 230A is coupled to trend analysis system 235A. Trend analysis system 235A is coupled to therapy system 240A which is, in turn, coupled to therapy electrode 250. Rate classification system 222A is further coupled to therapy system 240A.

In the figure, section 260A includes template 225A, similarity metric analysis system 220A, data processing system 230A, trend analysis system 235A, rate classification system 222A and therapy system 240A. In one embodiment, section 260A includes a microprocessor executing a program, along with memory and a pulse generator.

Cardiac electrode 205, lead 210 and sensing system 215 include devices and circuitry for receiving and sensing electrocardiogram signals. In addition, therapy electrode 250 and lead 245 include circuitry and devices for delivering therapy which may include an electrical pulse, electrical stimulation, therapeutic shock or other treatment. In one embodiment, a single electrode and a single lead, along with the housing of the ICD, both receives and senses ECG signals and delivers therapy and a switch coupled to the common lead selects between similarity metric analysis system 220A or therapy system 240A.

Template 225A, in one embodiment, includes a memory adapted for storing digital data corresponding to an NSR complex. The memory may include random access memory (RAM) or read only memory (ROM). In one embodiment, the data stored in template 225A is derived from historical data specific to the user. In one embodiment, the data stored in template 225A is based on model parameters. In one embodiment, the template data is collected during a normal sinus rhythm or during a rhythm that has normal conduction from atrium to ventricle

Similarity metric analysis system 220A, in one embodiment, includes a processor with programming adapted for generating a similarity metric, such as a correlation coefficient, based on a comparison of the signal received from cardiac electrode 205 and template 225A. The correlation may be expressed as a ratio or percentage or other measure. For example, the similarity metric, may be calculated by direct comparison of the template with the cardiac complex following alignment of a predetermined feature or characteristic of the template and the cardiac complex. The documents cited above provide exemplary methods of calculating a correlation coefficient.

Data processing system 230A, in one embodiment, calculates a moving average based on the series of correlation coefficients for each heart beat. For example, as each heart beat is sensed, a weighted average is calculated based on the correlation coefficient for that heart beat. In one embodiment, the correlation coefficient for the previous heart beats is weighted by a factor of 0.875 and the last heart beat is weighted by a factor of 0.125. The sum of the weighted correlation coefficients provides a moving average. In one embodiment, the maximum correlation coefficient values are discarded before calculating a moving average based on the ten most recent correlation coefficients. In one embodiment, the minimum correlation coefficient values are discarded before calculating a moving average based on the ten most recent correlation coefficients. Other means of processing the data are also contemplated. For example, a median or other function may be calculated and used as a metric for determining the trend exhibited by the cardiac complex. In addition, a smoothing function may be executed by data processing system 230A for purposes of reducing errors and improving accuracy of the analysis.

Data processing system 230A may be tailored to reduce artifacts of the correlation calculation process. For example, in one embodiment, the correlation analysis system samples the cardiac complex at a 200 Hz sampling rate. If a sample point does not align with a peak of the cardiac complex, or other feature of the cardiac complex, then the correlation coefficient for that heart beat may be inaccurate. In one embodiment, the data processing system is limited by the capacity of the processor and by practical limitations as to the time for delivery of a subsequent electrical stimulation if appropriate.

In one embodiment, data processing system 230A is omitted and the output from similarity metric analysis system 220A is provided directly to trend analysis system 235A. In one embodiment, trend analysis system 235A executes programming with sufficient rapidity to allow delivery of appropriate therapy to the heart. Trend analysis system 235A, in one embodiment, includes programming to determine if the correlation coefficient, based on the cardiac complex and the template, exhibits an increasing correlation.

Rate classification system 222A includes programming and circuitry to identify a cardiac rate based on a classification system. In one embodiment, rate classification system 222A may provide a signal to therapy circuit 240A calling for therapy to be delivered via therapy electrode 250A at a time when trend analysis system 235A also calls for therapy to be delivered or at a time when trend analysis system 235A calls for inhibiting therapy.

In one embodiment, 260A includes detection enhancing programming and circuitry. Detection enhancing programming and circuitry provides systems and methods for improving delivery of cardiac therapy based on receipt and analysis of cardiac signals.

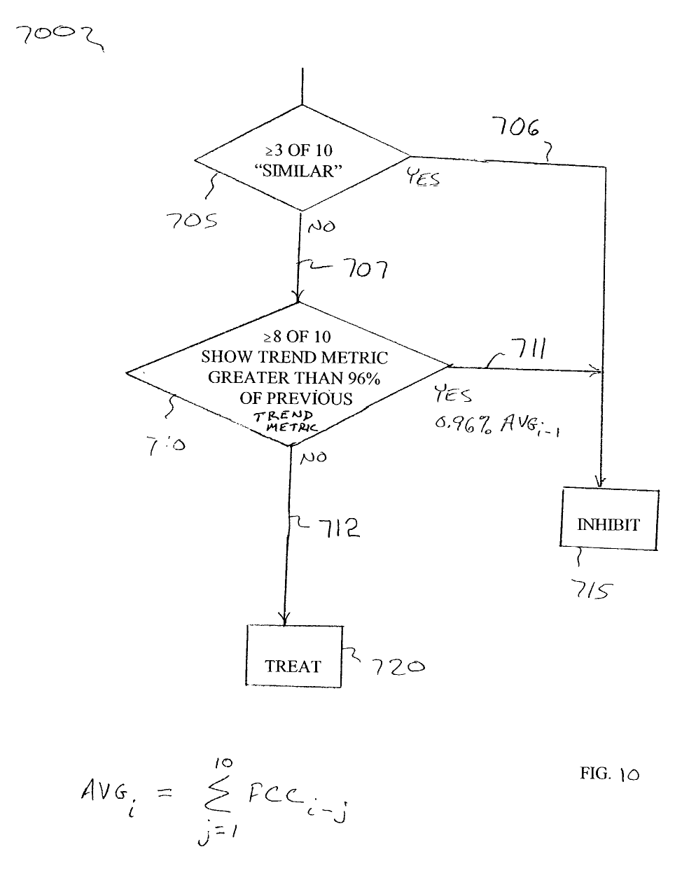

In one embodiment, arbitration between inconsistent signals received from trend analysis system 235A and rate classification system 222A is determined by deference to trend analysis system 235A. In one embodiment, if rate classification system 222A and trend analysis system 235A both call for treatment, then treatment is delivered and if either rate classification system 222A or trend analysis system 235A call for inhibiting treatment, then treatment is inhibited. FIG. 10 illustrates a rate classification system and a trend analysis system in one embodiment.

Therapy system 240A may include a pulse generator, a cardioverter, a defibrillation circuit or other system adapted to deliver an electrical stimulation. In one embodiment, a signal received by therapy system 240A, and provided by trend analysis system 235A, data processing system 230A or similarity metric analysis system 220A, controls the administration of an electrical stimulation.

Therapy electrode 250, in one embodiment, includes a conductive element coupled to the heart and adapted to deliver an electrical stimulation. In one embodiment, a single electrode provides electrical stimulations and also receives cardiac complexes. In one embodiment, one or more electrodes provides electrical stimulations and one or more electrodes receives cardiac complexes. In the figure, therapy electrode 250 is coupled to therapy system 240A.

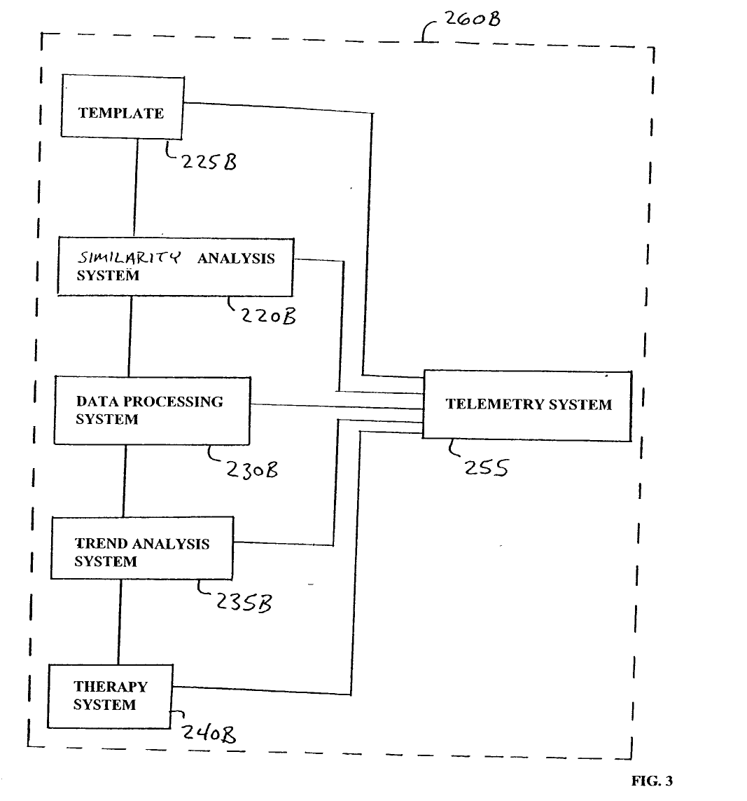

FIG. 3 illustrates section 260B having telemetry system 255 coupled to template 225B, similarity analysis system 220B, data processing system 230B, trend analysis system 235B and therapy system 240B. In the figure, telemetry system 255 is in communication with an external programmer or other device adapted for controlling or monitoring the operation of the present subject matter. For example, in one embodiment, telemetry system 255 receives correlation data from similarity analysis system 220B and communicates the data to an external programmer. As another example, in one embodiment, the external programmer can communicate an instruction to the present subject matter, via telemetry system 255, to invoke a replacement template 225B for comparison with future cardiac complexes. In one embodiment, telemetry system 255 is adapted to configure parameters with respect to a therapy algorithm. In various embodiments, any or all of the elements of section 260B are adapted for communicating data with an external programmer via telemetry system 255.

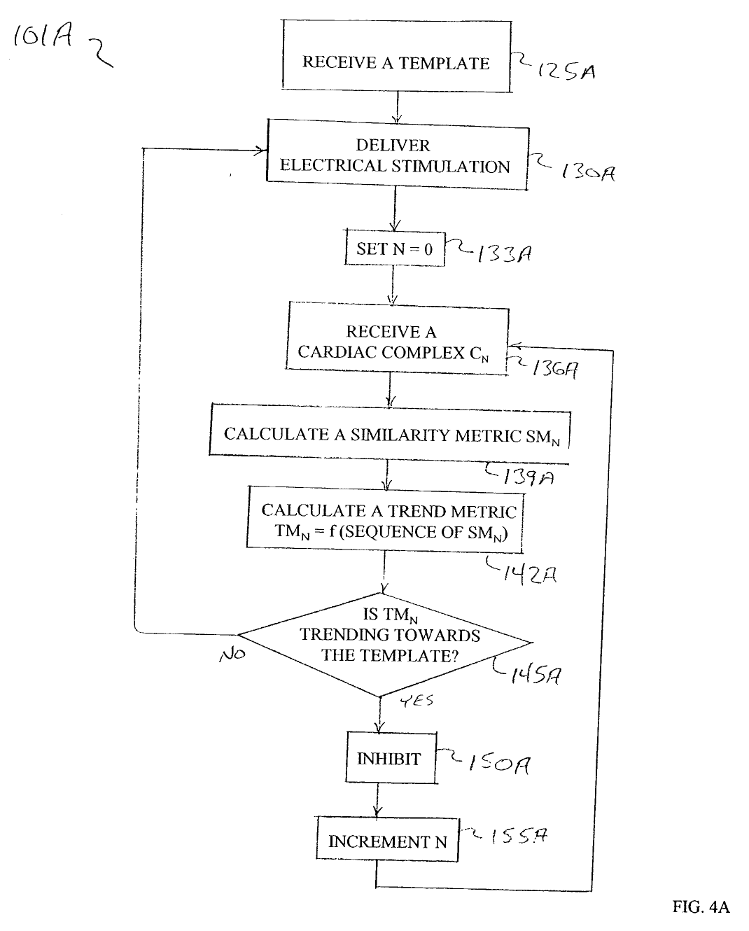

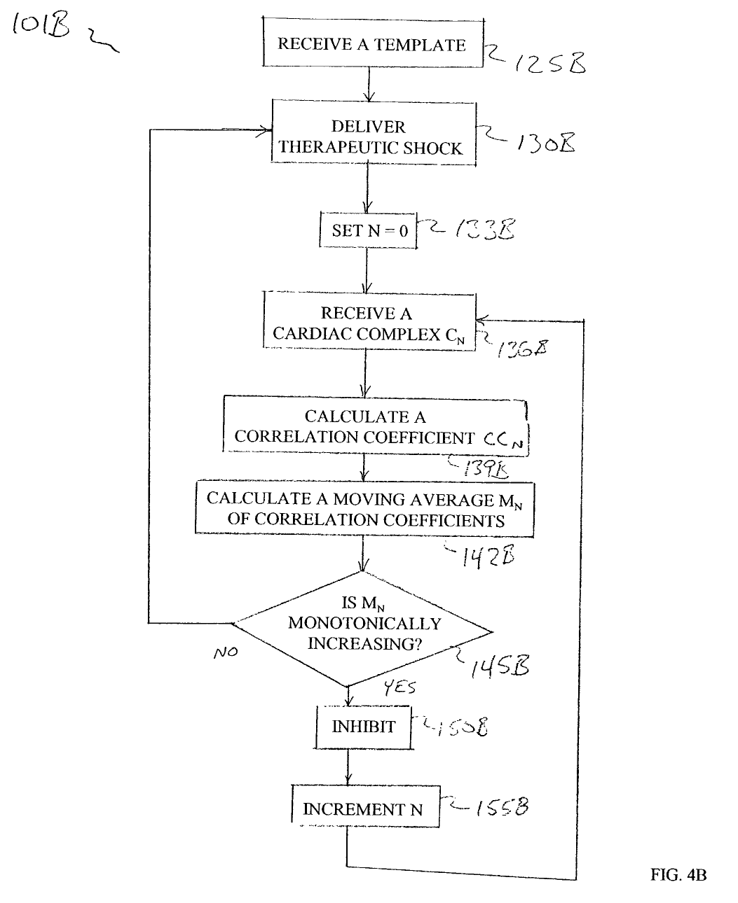

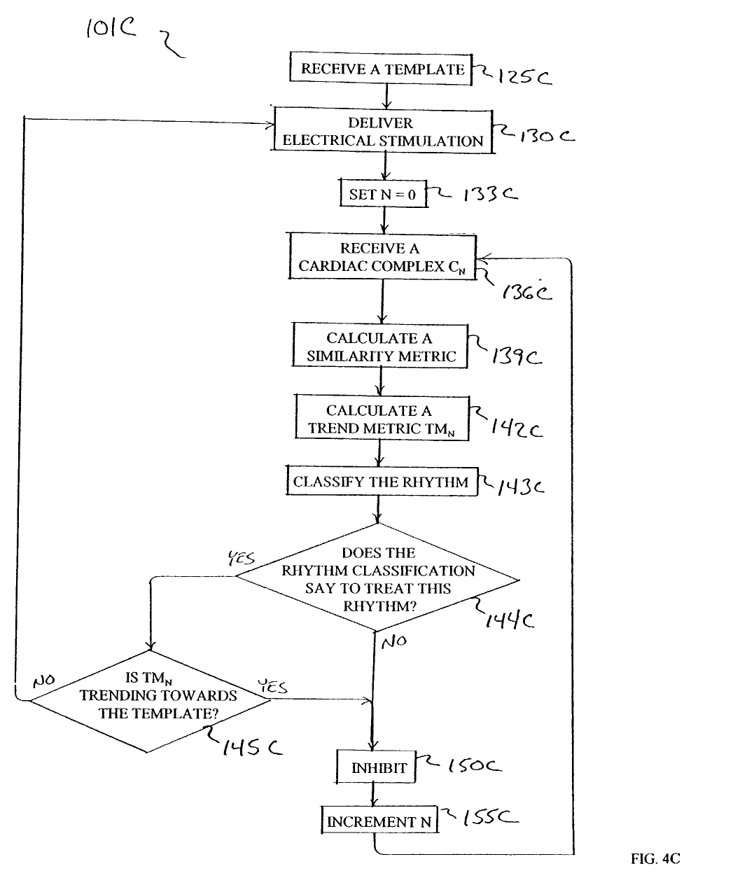

FIG. 4A illustrates method 101A performed by one embodiment of the present subject matter. At 125A, method 101A includes receiving a template. The template may include data representative of a model NSR or may include data derived from cardiac complexes during normal rhythm. In one embodiment, the template is updated from time to time. At 130A, an electrical stimulation is delivered. At 133A, a counter N is set to a value of zero. At 136A, the method includes receiving a cardiac complex, herein denoted as C_{N}, at a time following delivery of the electrical stimulation. At 139A, the method includes calculating a similarity metric, herein denoted as SM_{N}, based on a comparison of the template and cardiac complex C_{N}. At 142A, the method includes calculating trend metric TM_{N} where TM_{N} is a function of SM_{N} and TM_{N-1} (an earlier trend metric). In one embodiment, calculation of TM_{N} includes a smoothing function or averaging function. In one embodiment, TM_{N} is defined by (0.875)(TM_{N-1})+(0.125)(SM_{N}). At 145A, method 101A includes a query to determine if TM_{N} is trending towards the template. If, at the predetermined beat time, TM_{N} is not trending towards the template, then the method returns to 130A and an electrical stimulation is delivered. If, on the other hand, at the predetermined beat time, TM_{N} displays a trend towards the template, then therapy is inhibited, as denoted at 150A, the value of counter N is incremented, and the method continues with receipt of a subsequent cardiac complex C_{N}.

Variations in this method are also contemplated. For example, in one embodiment, the method omits setting and incrementing a counter and includes, at predetermined intervals, administering therapy based on metric TM_{N}. In one embodiment, a processor is adapted to execute a set of instructions to implement a method as shown in FIG. 4A.

FIG. 4B illustrates method 101B performed by one embodiment of the present subject matter. At 125B, method 100B includes receiving a template. At 130B, a therapeutic shock is delivered. At 133B, a counter N is set to a value of zero. At 136B, the method includes receiving a cardiac complex, herein denoted as C_{N}, at a time following delivery of the electrical stimulation. At 139B, the method includes calculating a correlation coefficient, herein denoted as CC_{N}, based on a comparison of the template and cardiac complex C_{N}. At 142B, the method includes calculating a moving average M_{N} based on a series of correlation coefficients. The moving average is indicative of a trend and M_{N} is a function of CC_{N} and M_{N-1} (a moving average correlation coefficient for a prior complex). In one embodiment, calculation of M_{N} includes a smoothing function or averaging function. In one embodiment, M_{N} is defined by (0.875)(M_{N-1})+(0.125)(CC_{N}). At 145B, method 100B includes a query to determine if M_{N} is increasing monotonically at the predetermined beat time. If, at the predetermined beat time, M_{N} does not exhibit a monotonic increase, then the method returns to 130B and a therapeutic shock is delivered. If, on the other hand, at the predetermined beat time, M_{N} displays a monotonic increase, then therapy is inhibited, as denoted at 150B, the value of counter N is incremented, and the method continues with receipt of a subsequent cardiac complex C_{N}.

FIG. 4C illustrates method 101C performed by one embodiment of the present subject matter. At 125C, method 101C includes receiving a template. At 130C, a therapeutic shock is delivered. At 133C, a counter N is set to a value of zero. At 136C, the method includes receiving a cardiac complex, herein denoted as C_{N}, at a time following delivery of the electrical stimulation. At 139C, the method includes calculating a similarity metric based on a comparison of the template and cardiac complex C_{N}. At 142C, the method includes calculating a trend metric TM_{N} based on a series of similarity metrics. The trend metric is indicative of a trend and TM_{N} is a function of the similarity metric for two or more prior complexes. In one embodiment, calculation of TM_{N} includes a smoothing function or averaging function.

At 143C, the rhythm is classified according to circuitry or an algorithm executing on a processor. The classification of the rhythm, in one embodiment, includes detection enhancement technology to determine if therapy is to be delivered.

At 144C, an inquiry is presented to determine if the rhythm classification system calls for delivery of therapy. If therapy is not prescribed, then processing continues at 150C where therapy is inhibited.

In the event that therapy is prescribed, then processing continues at 145C where an inquiry is presented to determine if the trend metric TM_{N} indicates a trend towards the template. If TM_{N} indicates that the cardiac complex is not trending towards the template, then processing continues at 130C where electrical stimulation is delivered. If TM_{N} indicates a monotonic increase, or otherwise indicates that a trend towards the template exists, then therapy is inhibited at 150C, counter N is incremented at 155C and processing continues at 136C where another cardiac complex is received.

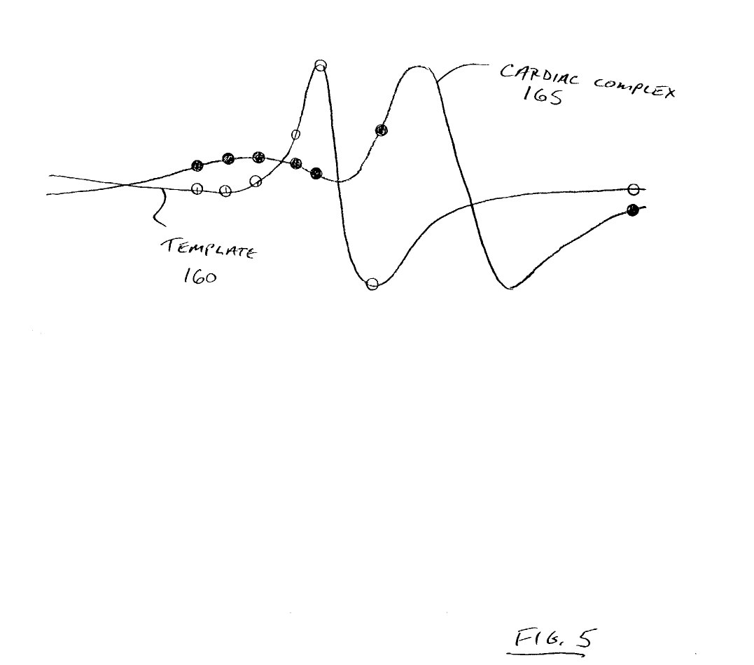

FIG. 5 graphically illustrates a time domain view of correlation between template 160 and cardiac complex 165. In general, the figure shows a lack of correlation, or a relatively low value of correlation. Feature amplitudes at eight predetermined locations on template 160 are compared to eight corresponding amplitudes of cardiac complex 165. As illustrated, the amplitude of the complex at each marked location is dissimilar to the amplitude of the template at the corresponding marked location and thus, the two signals are generally not correlated.

In determining the correlation, one embodiment provides that the cardiac complex is sampled at a frequency of 200 Hz. Sampling frequencies greater or less than 200 Hz are also contemplated.

Other means of evaluating correlation are also contemplated. For example, correlation may be determined by way of analysis performed in the frequency domain, rather than in the time domain.

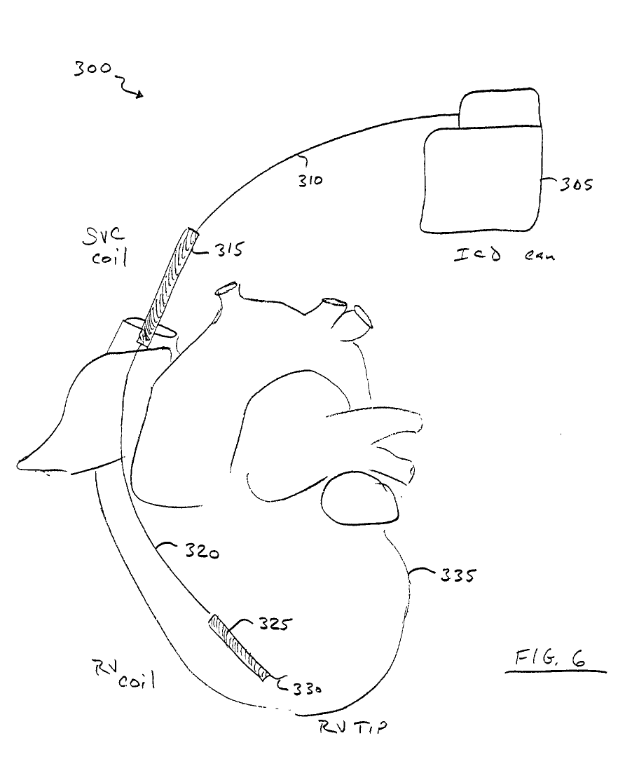

FIG. 6 illustrates system 300 with heart 335 coupled to one embodiment of the present subject matter, here denoted as ICD 305. Device 200, as described relative to FIG. 2, is housed within ICD 305. ICD 305 includes a conductive housing and is adapted for implantation within a chest cavity of a user. ICD 305 is electrically coupled to heart 335 by electrode 315 via lead 310. In addition, electrodes 325 and 330 within heart 335 are coupled to ICD 305 by lead 320. System 300, as illustrated in the figure, includes ICD 305 and electrodes 315, 325 and 330 and leads 310 and 320. In one embodiment, a single electrode is coupled to heart 335 and both receives an ECG signal and provides an electrical stimulation.

Other leads or electrodes are also contemplated. For example, in one embodiment, a remote sensing electrode is utilized for receiving cardiac complexes and an implanted electrode is utilized for delivering therapy. In one embodiment, multiple electrodes are used for receiving cardiac complexes.

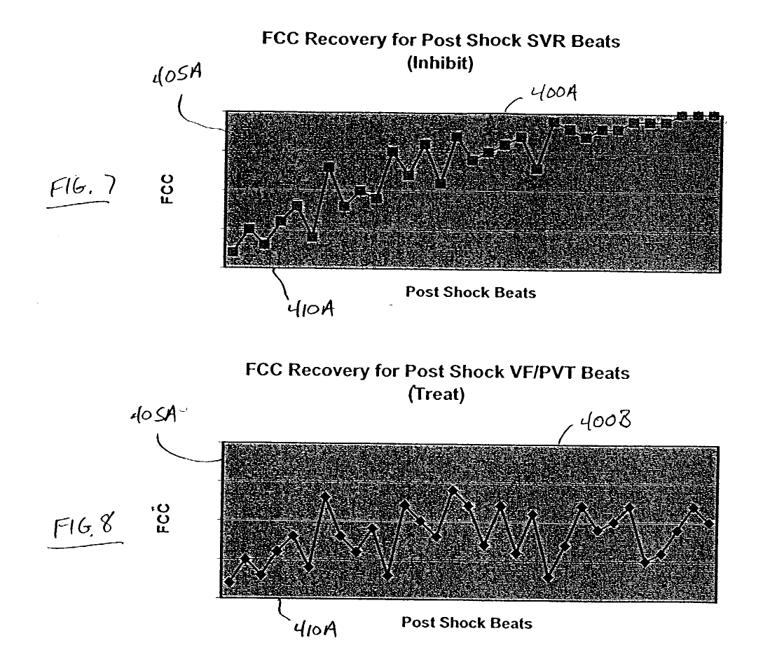

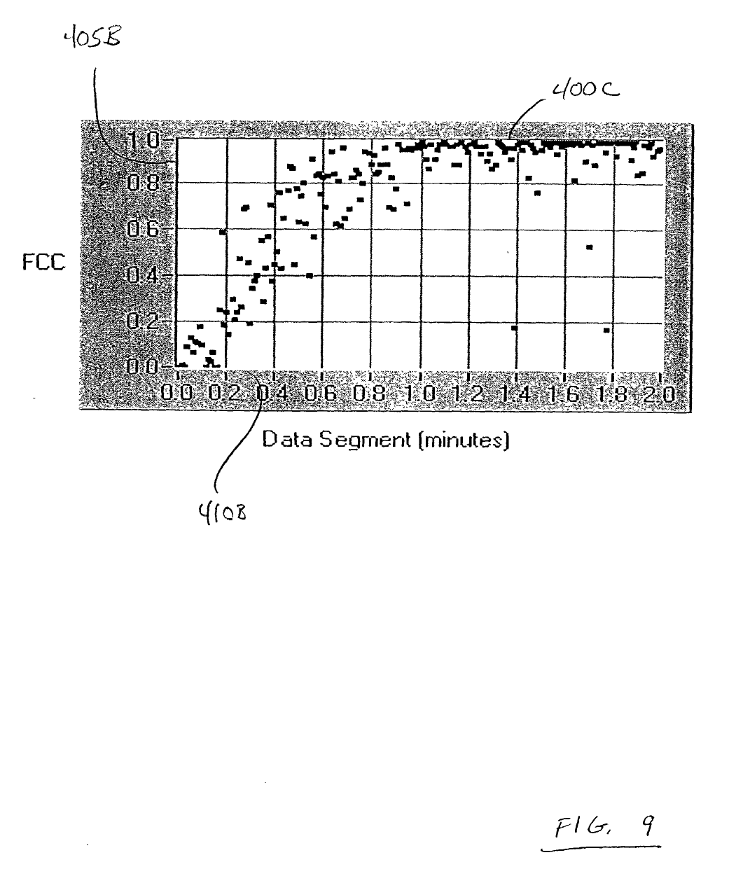

FIGS. 7 and 8 illustrate data showing an FCC plotted along the y-axis 405A, as a function of heart beats plotted along x-axis 410A with increasing time proceeding from left to right. The FCC is calculated based on the correlation between the cardiac complex and the template. An electrical stimulation was delivered at a time prior to, or simultaneous with, the first heart beat. In FIG. 7, for example, the average FCC is monotonically rising from a low value to a high value over the course of the 31 beats shown. In FIG. 8, on the other hand, the average FCC does not reflect a rising trend. Thus, for data 400A, shown in FIG. 7, the present subject matter is adapted to inhibit delivery of an electrical stimulation whereas, for data 400B, shown in FIG. 8, an electrical stimulation would be delivered at a predetermined time. In FIG. 7, the feature correlation coefficient appears to be asymptotically trending towards the template.

In FIG. 9, the FCC ranges between 0.0 and 1.0 along y-axis 405B and x-axis 410B depicts a two minute interval of time following delivery of an electrical stimulation. As illustrated, each data point corresponds to a feature correlation coefficient calculated at the indicated times. An averaging function is applied to the data to aid in discerning the trend towards agreement with the template. The averaging function, for example, suppresses anomalous data points, including those exemplary data points just prior to 1.4 minutes and just prior to 1.8 minutes.

FIG. 10 illustrates a portion of one method according to the present subject matter. In one embodiment, an FCC (or other similarity metric) is calculated by comparing a cardiac complex to a template, as shown at 705. At 706, if three or more of the ten most recent FCC's are similar, then inhibit. A cardiac complex is similar if it is correlated to a template by more than 94%. In one embodiment, the similarity metric is calculated using an autoregressive average function.

Next, at 710, the inquiry determines if the trend metric indicates a trend towards the template. At 711, if eight or more out of ten most recent beats have a trend metric that is greater or equal to 96% of the previous trend metric, then therapy is inhibited as shown at 715. If, on the other hand, and as shown at 712, eight out of ten beats do not have a trend metric greater or equal to 96% of the previous trend metric, then treatment ensues and therapy is delivered as shown at 720. In one embodiment, the trend metric includes an average correlation coefficient based on a sliding ten beat window. For example, in one embodiment, the average is the summation of the previous ten FCC values and if the average for one particular cardiac complex is greater than 96% of the average for the previous cardiac complex for at least eight out of the last ten beats, then continue to inhibit, otherwise, if less than three of 10 are similar, then treat. FIG. 10 illustrates a method for executing this routine. On each new beat, inquiry 705 determines if the beat is similar for at least three of the ten matches. In one embodiment, this entails determining if the correlation coefficient is greater or equal to 94% for at least three of the last ten beats.

In one embodiment, for purposes of computational simplicity, the correlation coefficient is defined to fall in the range of −1 to +1 with negative values corresponding to uncorrelated data. Negative correlation is to be treated as data dissimilar to the template.

Alternative Embodiments

Variations of the above embodiments are also contemplated. For example, in one embodiment, the correlation between the cardiac complex and the template is determined after a transform is executed. The transform may include a Fourier transform to convert the time domain data of the cardiac complex into the frequency domain.

In one embodiment, a processor of the present subject matter is adapted to determine the dissimilarity between the cardiac complex and the template and execute a program to deliver therapy in the event that the dissimilarity is increasing in a particular manner.

In one embodiment, the moving correlation coefficient is used to administer therapy without calculating an average value. In this embodiment, the processor executes programming instructions with sufficient speed to meet the requirements of timely delivery of therapy.

In one embodiment, delivery of therapy includes delivering electrical stimulation therapy synchronized with delivery of an intrinsic ventricular wave or R wave. Synchronization of the electrical stimulation therapy and the R wave may be within 50 milliseconds.

A programmer, in wired or wireless communication with the present subject matter, can be used to access stored data corresponding to detected cardiac complexes. In one embodiment, the stored data indicates whether therapy was delivered or therapy was inhibited for a particular episode of cardiac complexes. For example, in one embodiment modeled by FIG. 4A, when therapy is inhibited at 150A, data is stored in a memory of the present subject matter indicting that the rhythm corresponds to SVT. As another example, and with reference to FIG. 10, in one embodiment, when therapy is inhibited, as at 715, data is stored in a memory of the present subject matter indicating that the rhythm corresponds to SVT and when therapy is delivered, or treatment is delivered, as at 720, data is stored indicating that the rhythm corresponds to VT or VF. A rhythm corresponding to SVT indicates tachycardia having a ventricular origin. A rhythm corresponding to VT or VF indicates tachycardia having an atrial origin.

### CONCLUSION

The above description is intended to be illustrative, and not restrictive. Many other embodiments will be apparent to those of skill in the art upon reviewing the above description.

What is claimed is:

1. A method comprising:
receiving a cardiac complex following an electric stimulation;
calculating a similarity metric based on the cardiac complex and a stored template;
calculating a trend metric based on the similarity metric and an earlier similarity metric; and
inhibiting therapy if the trend metric indicates that the cardiac complex is approaching the template.

2. The method of claim 1 further comprising storing data indicating that the cardiac complex corresponds to a tachycardia having an atrial origin.

3. The method of claim 1 wherein receiving a cardiac complex includes receiving a plurality of signals from a plurality of electrodes.

4. The method of claim 1 wherein receiving the cardiac complex following an electric simulation includes receiving the cardiac complex following a therapeutic shock.

5. The method of claim 1 further comprising delivering therapy if the trend metric is not monotonically increasing.

6. The method of claim 1 further comprising delivering therapy if the trend metric is not monotonically increasing at a time synchronized with an R-wave.

7. The method of claim 1 further comprising delivering therapy if the trend metric is not monotonically increasing at a time synchronized within 50 milliseconds of an R-wave.

8. The method of claim 1 wherein calculating the similarity metric includes calculating a correlation coefficient.

9. The method of claim 1 wherein calculating the similarity metric includes calculating a correlation coefficient in a time domain.

10. The method of claim 1 wherein calculating the similarity metric includes sampling the cardiac complex.

11. The method of claim 1 wherein calculating the trend metric based on the similarity metric and the earlier similarity metric includes calculating a moving average based on the similarity metric and a plurality of earlier similarity metrics.

12. The method of claim 11 wherein calculating the trend metric includes discarding one or more selected similarity metrics of the plurality of similarity metrics.

13. The method of claim 11 wherein calculating the trend metric includes, for every ten similarity metrics of the plurality of similarity metrics, discarding one or more selected similarity metrics.

14. The method of claim 11 further including:
selecting a first subset of similarity metrics from the plurality of earlier similarity metrics; and
wherein calculating the similarity metric includes calculating a correlation coefficient; and
wherein calculating the trend metric based on the similarity metric and the earlier similarity metric includes calculating the trend metric based on the similarity metric and the first subset of similarity metrics.

15. A method comprising:
detecting an electrical stimulation;
receiving a first cardiac complex and a second cardiac complex after the electrical stimulation;
calculating a first similarity metric based on the first cardiac complex and a template;
calculating a second similarity metric based on the second cardiac complex and the template;
calculating a trend metric based on the first similarity metric and the second similarity metric; and
inhibiting therapy if the trend metric indicates a trend towards the template.

16. The method of claim 15 wherein calculating a first similarity metric includes calculating a first correlation coefficient and calculating a second similarity metric includes calculating a second correlation coefficient.

17. The method of claim 15 wherein receiving a first cardiac complex and a second cardiac complex includes receiving a plurality of signals from a plurality of electrodes.

18. The method of claim 15 wherein calculating the first similarity metric and calculating the second similarity metric includes sampling the cardiac complex.

19. A method comprising:
receiving a template complex;
receiving a series of cardiac complexes occurring at a time following a first electrical stimulation;
calculating a series of similarity metrics wherein each similarity metric is determined based on a comparison of a cardiac complex of the series of cardiac complexes and the template complex;
calculating a series of trend metrics wherein each trend metric is determined based on a plurality of similarity metrics of the series of similarity metrics;
evaluating the series of trend metrics to determine if the series of cardiac complexes is trending towards the template complex; and
administering therapy based on the evaluating.

20. The method of claim 19 further comprising classifying the series of cardiac complexes.

21. The method of claim 20 further comprising administering therapy based on classifying the series of cardiac complexes.

22. The method of claim 19 wherein calculating the similarity metric includes calculating a correlation coefficient.

23. The method of claim 19 wherein calculating the trend metric includes calculating an average.

24. The method of claim 19 wherein calculating the trend metric includes calculating a moving average.

25. The method of claim 19 wherein calculating the trend metric includes calculating a moving average of four similarity metrics.

26. The method of claim 19 wherein calculating the trend metric includes calculating a moving average of ten similarity metrics.

27. The method of claim 19 wherein evaluating the series of trend metrics includes comparing a first trend metric with a product of a factor and a second trend metric.

28. The method of claim 19 wherein administering therapy includes delivering a second electrical stimulation.

29. The method of claim 19 wherein administering therapy includes storing data indicating that the series of cardiac complexes corresponds to a tachycardia having a ventricular origin.

30. The method of claim 19 wherein administering therapy includes delivering a second electrical stimulation if the series of trend metrics corresponds to a non-monotonically increasing trend over a predetermined period of time.

31. The method of claim 19 wherein administering therapy includes inhibiting therapy if a subset of the series of trend metrics corresponds to a monotonically increasing trend over a predetermined period of time.

32. A method comprising:
receiving a series of cardiac complexes at a time occurring after a first electrical stimulation and before a second electrical stimulation, the second electrical stimulation subsequent to the first electrical stimulation;
for a predetermined period of time following the first electrical stimulation, generating a series of correlation coefficients based on a comparison of the series of cardiac complexes and a stored template;
calculating a series of trend metrics for the series of correlation coefficients, each trend metric based on a plurality of correlation coefficients selected from the series of correlation coefficients; and
inhibiting delivery of the second electrical stimulation if a predetermined subset of the series of trend metrics indicates that the series of cardiac complexes is approaching the template.

33. The method of claim 32 wherein inhibiting delivery of the second electrical stimulation if the predetermined subset of the series of trend metrics indicates that the series of cardiac complexes is approaching the template includes inhibiting delivery of the second electrical stimulation if, for each of three selected correlation coefficients of a series of ten correlation coefficients, the selected correlation coefficient exceeds a predetermined value.

34. The method of claim 33 wherein the predetermined value is 94 percent.

35. The method of claim 32 wherein inhibiting delivery of the second electrical stimulation if the predetermined subset of the series of trend metrics indicates that the series of cardiac complexes is approaching the template includes inhibiting delivery of the second electrical stimulation if, for each of eight selected trend metrics of a series of ten trend metrics, the selected trend metric exceeds a product of a prior trend metric and a factor.

36. The method of claim 35 wherein the factor is 96 percent.

37. A system comprising:
a sensing means adapted to couple to a cardiac electrode means and adapted to detect a series of cardiac complexes;
a correlation analysis means coupled to the sensing means and adapted to generate a series of correlation coefficients based on the series of cardiac complexes and a template;
a trend analysis means coupled to the correlation analysis means and adapted to analyze a trend based on the series of correlation coefficients; and
a therapy delivery means coupled to the trend analysis means and adapted to deliver therapy based on a signal received from the trend analysis means.

38. The system of claim 37 further comprising a data processing means coupled to the correlation analysis means and adapted to execute a function using the series of correlation coefficients and provide a signal to the trend analysis means.

39. The system of claim 37 further comprising a therapy electrode means coupled to the therapy delivery means and adapted to deliver an electrical stimulation.

40. The system of claim 37 further comprising a telemetry means coupled to, and adapted to communicate with, the correlation analysis means, the trend analysis means, the data processing means or the therapy delivery means.

41. A method comprising:
receiving a series of cardiac complexes at a time following an electrical stimulation;
generating a series of correlation coefficients, each correlation coefficient based on a comparison of a cardiac complex with a stored template;
evaluating a subset of the series of correlation coefficients to determine if a monotonically increasing trend exists; and
inhibiting therapy if the monotonically increasing trend exists.

42. The method of claim 41 wherein evaluating a subset of the series of correlation coefficients includes discarding one or more maximum correlation coefficients.

43. The method of claim 41 wherein evaluating a subset of the series of correlation coefficients includes discarding one or more minimum correlation coefficients.

44. The method of claim 41 wherein inhibiting therapy includes inhibiting delivery of a second electrical stimulation.

45. The method of claim 41 wherein evaluating the subset of the series of correlation coefficients includes evaluating ten correlation coefficients.

46. The method of claim 41 further comprising calculating a series of trend metrics based on the series of correlation coefficients and wherein inhibiting therapy if the monotonically increasing trend exists includes inhibiting therapy if each of eight trend metrics of a group of ten trend metrics in the series of trend metrics exceeds a product of a prior trend metric and a factor.

47. The method of claim 46 wherein the factor is 96 percent.

48. A method comprising:
receiving a cardiac complex following an electric stimulation;
calculating a similarity metric based on the cardiac complex and a stored template;
calculating a trend metric based on the similarity metric and an earlier similarity metric; and
storing data indicating that the cardiac complex corresponds to a tachycardia having an atrial origin if the trend metric indicates that the cardiac complex is approaching the template.
